(19) United States
(12) Patent Application Publication
(10) Pub. No.: US 2016/0030163 A1
(43) Pub. Date: Feb. 4, 2016

(54) Surgical Apparatus and Method Of Implanting The Same

(71) Applicant: Art, Limited, Grand Cayman (KY)

(72) Inventor: Takayuki Akahoshi, Tokoyo (JP)

(21) Appl. No.: 14/813,800

(22) Filed: Jul. 30, 2015

Related U.S. Application Data
(60) Provisional application No. 62/031,451, filed on Jul. 31, 2014.

(51) Int. Cl. A61F 2/16

(52) U.S. Cl. CPC A61F 2/1662; A61F 2/16; A61F 2002/1681; A61F 2002/169; A61F 2002/16903

(57) ABSTRACT

A surgical apparatus is provided for implantation into organic tissue for use with an implant having one or more stabilizing haptics. The surgical apparatus has a ring-like body for stabilizing organic tissue. The body has an outer surface and one or more abutments extending from the outer surface. The abutment is for engaging the stabilizing haptics of the implant to inhibit movement of the implant within the organic tissue.

### FIELD OF THE INVENTION

The present invention relates generally to the field of surgery, and more particularly, to a surgical apparatus and method for implanting such an apparatus within organic tissue.

### BACKGROUND OF THE INVENTION

When the eye becomes aged, diseased, or injured it may be necessary to remove the natural lens of the eye. Such removal is common for cataract surgery, in which a lens that has become clouded is removed. The removal of the natural lens of the eye may result in the loss or alteration of focused vision of a patient. Therefore, an artificial lens may be necessary to restore the vision of the patient. Some eyes have an oblong, irregular shaped cornea that causes astigmatism, or blurred vision due to a refractive error in the eye.

Such artificial lenses may be provided in eyeglasses, contact lenses, or a permanent implant known as an intraocular lens (hereinafter “IOL”). The IOL is an artificial, generally circular lens with one or more stabilizing projections, arms, or haptics extending from the lens. A special type of implant called a toric IOL may be used to correct for astigmatism in the eye. The toric IOL has a lens body with an astigmatic axis that must be aligned with the steep corneal meridian of the eye. To implant the IOL in the eye, an incision is made in the anterior portion of the eye, typically while maintaining positive pressure within the eye to prevent collapse of the delicate structures of the eye. The IOL is generally folded or otherwise placed in a compressed state within an injector housing. The injector housing is elongate for being placed through the incision and into the patient's eye after the natural lens has been dismembered and aspirated, such as through phacoemulsification. A plunger is retained within the injector housing and is movable with respect to the housing. Movement of the plunger through the housing presses the IOL forward into the eye. The IOL, typically being resilient, will subsequently expand to an uncompressed state upon entering the eye and exiting the injector. The haptics of the IOL serve to balance and center the IOL within the eye of the patient. The IOL is typically made from biocompatible materials such as PMMA, silicone, or acrylic.

During the removal of the natural lens of the eye, a physician may note that the zonules, or supporting ligaments of the capsular bag which contains the lens, are weakened, deteriorated, or otherwise insufficient to provide adequate structural support to centralize the haptics of the IOL within the eye. Therefore, a capsular tension apparatus or ring may be required to exert an outward pressure on the capsular bag prior to implantation of the IOL. Such capsular tension rings are typically C-shaped and formed from a biocompatible material such as PMMA. Capsular tension rings may be inserted through an incision in the anterior portion of the eye or may be folded and injected into the incision in a similar fashion as described above with respect to the IOL.

To maintain the depth of the capsular bag during the implantation of the IOL, and thus maximize the ability of the surgeon to manipulate the anterior chamber throughout the implantation process, an ophthalmic viscoelastic device (hereinafter “OVD”) or material is used to fill the capsular bag. However, this OVD must be removed after the implantation of the IOL because it can lead to postoperative intraocular peaks in pressure. Removal of the OVD typically causes the IOL to shift or rotate in a clockwise direction due to the design of the haptics. For the toric IOL, such shifting may result in degradation of the quality of the vision of the patient and require costly corrective surgical procedures.

### SUMMARY OF THE INVENTION

The inventor of the present invention has discovered a plurality surgical apparatuses and methods for implanting such apparatuses that improve the stabilization and centering of an implanted IOL within the eye. It will be appreciated, however, that that novel apparatuses and methods for implanting the same are not limited to the field of ophthalmology and may be used in surgical operations on other areas of the human or non-human body.

In accordance with the present invention, a surgical apparatus is provided for implantation into organic tissue for use with implant having at least one stabilizing haptic extending therefrom. The apparatus has a ring-like body for stabilizing the organic tissue. The body has an outer surface and at least one abutment extending from the outer surface. The at least one abutment is for engaging the at least one haptic of the implant to inhibit movement of the implant within the organic tissue.

In accordance with another aspect of the present invention, a method is provided for implanting a surgical apparatus into organic tissue. The method has the steps of: (a) providing a surgical apparatus having a ring-like body for stabilizing the organic tissue, the body having an outer surface and at least one abutment extending from the outer surface; (b) providing an implant having at least one stabilizing haptic extending therefrom; (c) injecting a viscoelastic device into the organic tissue; (d) implanting the surgical apparatus within the organic tissue; (e) implanting the implant within in the organic tissue to engage the surgical apparatus; and (f) removing the viscoelastic device from the organic tissue such that movement of the implant within the organic tissue is inhibited by engagement of the at least one stabilizing haptic with the at least one abutment of the surgical apparatus.

The above features and advantages of the present invention will be better understood with reference to the accompanying figures and detailed description.

### BRIEF DESCRIPTION OF THE DRAWINGS

FIG. 1 is a cross-sectional diagrammatic view of a human eye;

FIG. 2 is a plan view of a prior art capsular tension ring;

FIG. 3 is a plan view of a prior art intraocular lens;

FIG. 4 is a diagrammatic plan view the prior art capsular tension ring of FIG. 2 and the intraocular lens of FIG. 3 shown implanted into a human eye;

FIG. 5 is a plan view of a first embodiment of a capsular tension apparatus according to the present invention;

FIG. 6 is a detailed, broken view of the capsular tension apparatus of FIG. 5;

FIG. 7 is a plan view of a novel intraocular lens with anti-rotation features;

FIG. 8 is a detailed perspective view of the intraocular lens of FIG. 7;

FIG. 9 is a diagrammatic plan view the capsular tension apparatus of FIG. 5 and the intraocular lens of FIG. 7 shown implanted into a human eye;

FIG. 10 is a plan view of another embodiment of a capsular tension apparatus according to the present invention;

FIG. 11 is a detailed, broken view of the capsular tension apparatus of FIG. 10;

FIG. 12 is a side elevational view of another embodiment of a capsular tension apparatus according to the present invention; and

FIG. 13 is a side elevational view of another embodiment of a capsular tension apparatus according to the present invention.

### DETAILED DESCRIPTION OF THE PREFERRED EMBODIMENTS

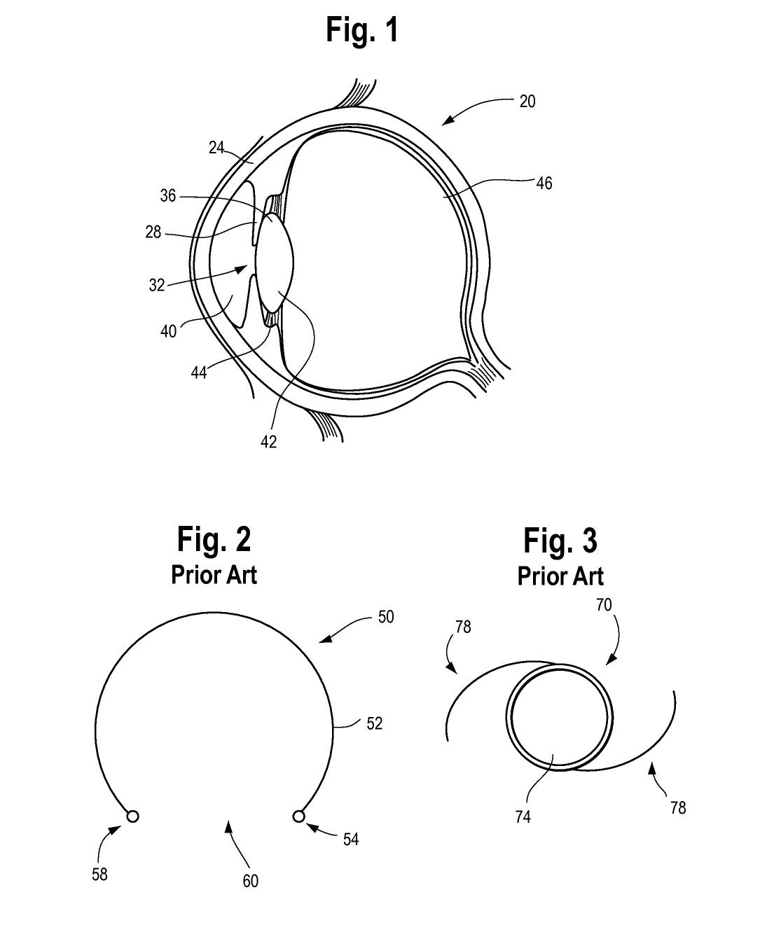

FIG. 1 shows a diagrammatic cross-sectional view of the human eye 20. Beginning at the exterior of the eye 20, the eye 20 has a protective outer layer or cornea 24 which retains the fluids or aqueous humor of the eye 20. Inward of the cornea 24 is the ring-like iris 28 with an aperture or pupil 32 for restricting light reaching the lens 36. The lens 36 is located within a front or anterior cavity 40 of the eye 20 and is encased in a capsular bag 42. Supporting ligaments or zonules 44 stabilize and center the capsular bag 42 within the eye 20. Opposing the anterior cavity 40 of the eye 20 is the posterior cavity 46 containing the optical nerves and arteries of the eye 20. When the lens 36 has been removed from the eye 20, such as through phacoemulsification, some or all of the capsular bag 42 remains connected to the zonules 44 in the eye 20.

FIG. 2 shows a typical prior art capsular tension apparatus or ring 50. The prior art capsular tension ring 50 is made from a biocompatible material such as PMMA and has a body 52 with a first end 54 and second end 58 that define an opening 60. The opening 60 results in the ring 50 having a generally C-shape and inherent degree of flexibility for manipulation by the surgeon implanting the capsular tension ring 50 into the capsular bag 42. The ends 54, 58 of the ring 50 have the form of an eyelet that provide a stable location for receiving a surgical tool to position the ring 50 within the eye 20. Ring 50 is typically sized between 12-13 mm in diameter.

FIG. 3 shows a typical prior art toric IOL 70. The prior art toric IOL 70 is also made from a biocompatible material such as PMMA, silicone, or acrylic and has a central lens body 74. The lens body 74 may have one or more surfaces of a varying degree of convexity depending on the need for correction to the patient's vision. The toric IOL has an astigmatic axis that must be aligned with the steep corneal meridian. Two generally arcuate arms or haptics 78 extend generally radially outward from the exterior surface of the lens body 74. The haptics 78 are intended to contact the capsular bag 42 to center the lens body 74 within the eye 20, as will be discussed in detail below.

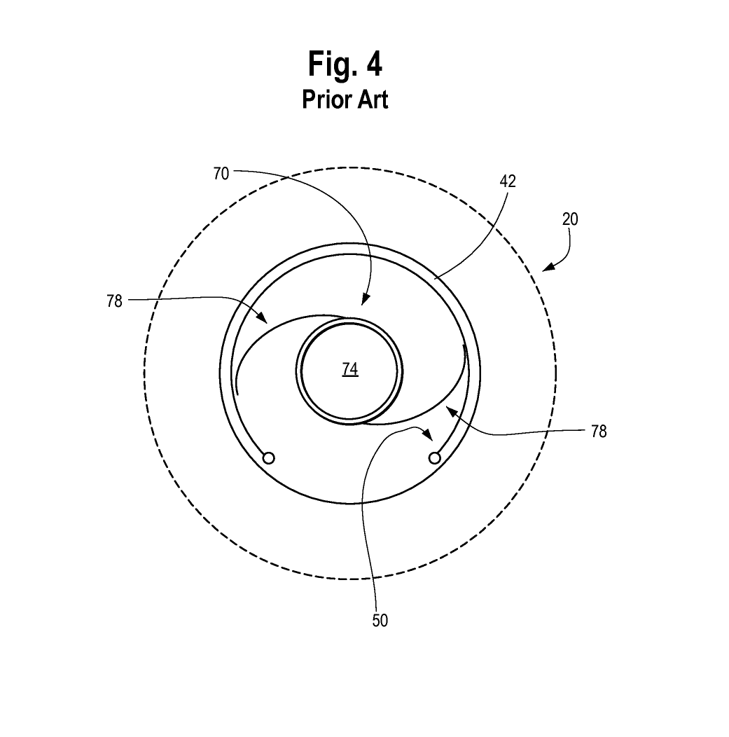

Referring now to FIG. 4, which is a diagrammatic plan view of prior art toric IOL 70 and prior art capsular ring 50 shown implanted in the eye 20. The ring 50 can be seen to support the periphery of the capsular bag 42 such that the haptics 78 touch the interior surface of the capsular bag 42 and/or ring 50 to center the lens body 74 of the toric IOL 70.

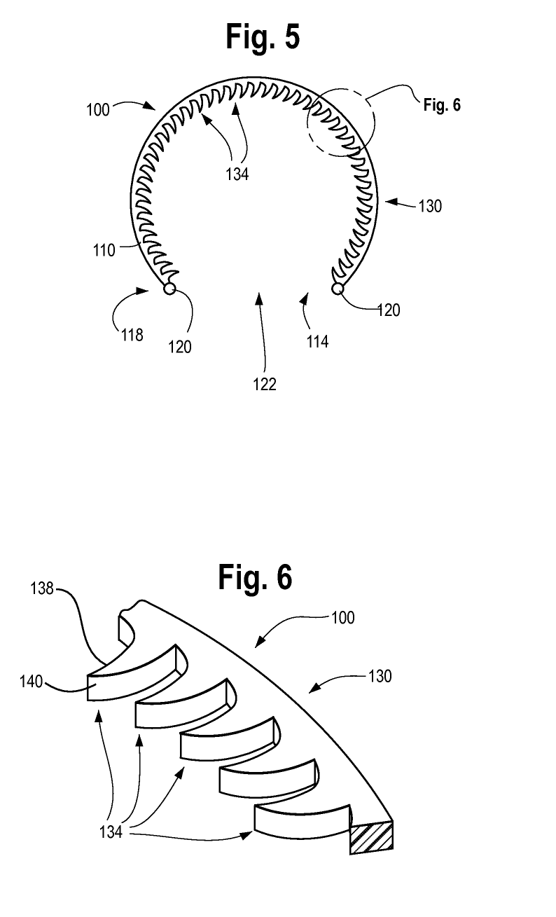

A first embodiment of an inventive surgical apparatus, capsular tension apparatus or simply “ring” 100 is illustrated in FIG. 5. The ring 100 is made from a biocompatible, semi-flexible material that is preferably compression-molded PMMA. The ring 100 has a body 110 and, in the preferred illustrated embodiment, the body 110 has a first end 114 and second end 118 that together define an opening 122. The opening 122 results in the ring 100 having a generally C-shape and inherent degree of flexibility for manipulation by the surgeon implanting the ring 100 into the capsular bag 42. The ends 114, 118 of the ring 100 may further have one or more platforms or eyelets 120 that provide a stable location for receiving a surgical tool to position the ring 100 within organic tissue or the eye 20. In the broadest concept of the invention, the ring 100 need not have any discernible ends, and may instead be comprised of a closed loop or polygon. Furthermore, the body 110 need not be a unitary structure and may instead be comprised of multiple sections connected or otherwise secured to one another to form the body 110.

FIG. 6 is a detailed view of an exterior or outer surface 130 of the ring 100.

Outer surface 130 is shown with a plurality of teeth or barb-like projections or abutments 134 extending generally radially inwardly toward the open center of the body 110. The abutments 130 are for engaging the movement inhibiting or anti-rotation projections of a novel intraocular lens (discussed in detail hereinafter) to inhibit movement of such a novel intraocular lens in the eye 20. In the broadest form of the present invention, the body 110 need not have a plurality of abutments 134 and may only have a single abutment 134.

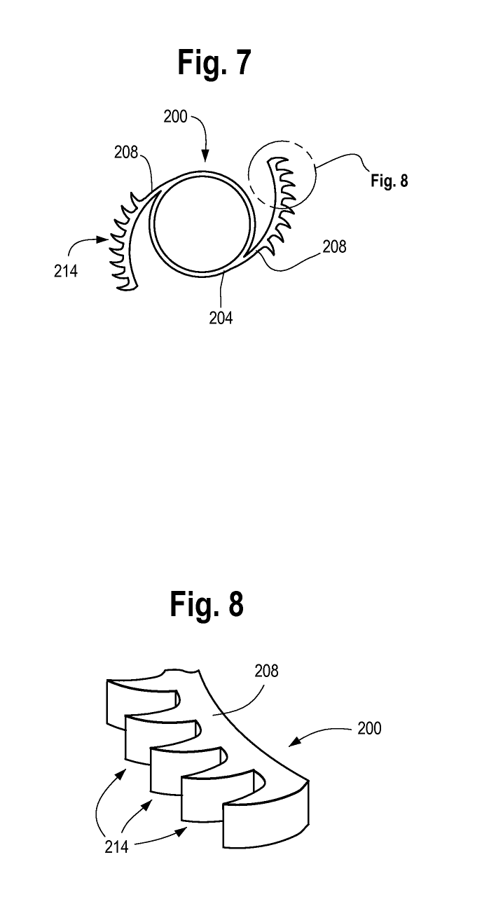

Referring next to FIGS. 7 and 8, an inventive toric IOL 200 is illustrated. The toric IOL 200 is made from a biocompatible material such as PMMA, silicone, or acrylic and has a central lens body 204. The lens body 204 may have one or more surfaces of a varying degree of convexity depending on the need for correction to the patient's vision. Two arms or haptics 208 extend generally radially outward from the exterior surface of the lens body 204. The haptics 208 are intended to stabilize the lens body 204. Furthermore, haptics 208 have one or more movement inhibiting or anti-rotation projections 214 extending generally radially outwardly from the haptics 208 to engage with the abutments 134 in order to center the lens body 204 within the eye 20, as will be discussed in detail below.

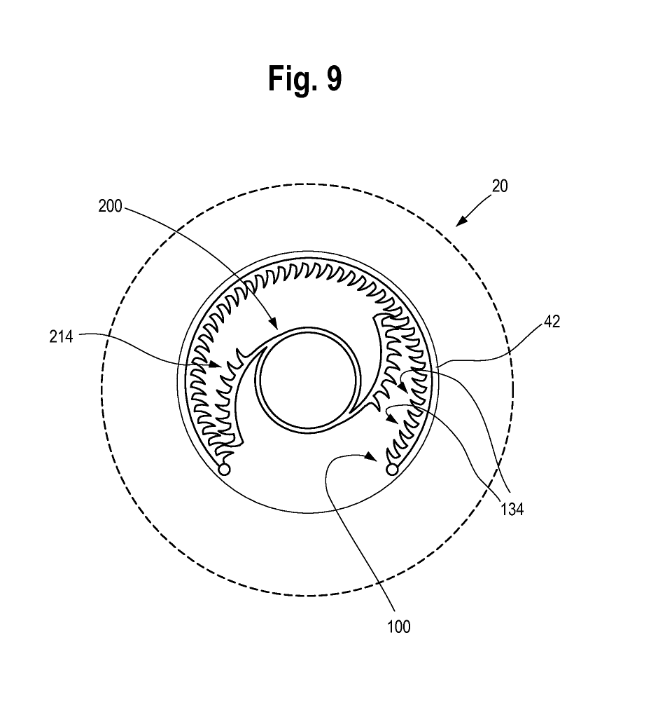

For the toric IOL 70, 200 a one degree of misalignment of the toric IOL 70, 200 astigmatic axis with the steep corneal meridian of the eye may result in a loss of over 3% of the toric effect (for correction of the astigmatism of the eye). Furthermore, rotational misalignment of the toric IOL astigmatic axis with the steep corneal meridian of the eye of over 30% may result in a total loss of toric effect, and may even induce astigmatism. The inventor has discovered that the ring 100 having one or more abutments 134 for engagement with one or more anti-rotation projections 214 of an inventive toric IOL 200 can inhibit undesirable operative and/or post-operative movement of the toric IOL 200, as shown in FIG. 9. However, the ring 100 may be used with prior art toric IOL 70 such that the abutments 134 engage the haptics 78. The abutments 134 serve to limit the susceptibility of the IOL 70, 200 to rotate, translate, or otherwise shift to a position that degrades the vision-correcting ability of the lens body 74, 204. By limiting such movement of the toric IOL 70, 200, the ring 100 and toric IOL 70, 200 together may reduce and/or eliminate undesirable rotation or misalignment of the toric IOL 70, 200.

The first illustrated embodiment of ring 100, in FIG. 6 shows the abutments 134 to have the form of a spike that is defined by two converging surfaces 138, 140 for preventing rotation of the toric IOL 200 in a clockwise direction. In the preferred embodiments of the ring 100, there may be eighteen spikes every ten degrees or thirty-six spikes every five degrees to limit the movement of the toric IOL 200.

In the broadest concept of the invention, however, the abutments 134 may take a variety of geometries such as straight or angled cantilevered beams projecting from any surface of the ring 100. Such beams may have shapes that are fully arcuate, partially arcuate, or polygonal. Furthermore, the abutments 134 may be comprised of hooks or loops for engaging anti-rotation projections of the toric IOL 200 that are reciprocal hooks or loops. The abutments 134 need not be unitarily formed with the ring body 110 and may be attached to the body 110 in a secondary process or made from a different biocompatible material. The abutments 134 need not be simple cantilevered bodies or evenly spaced on the ring body 110, and may instead be comprised of one or more skirts or united projections extending outwardly along the ring body 110. The abutments 134 need not be formed in the plane defined by the ring body 110 and may extend outwardly from such a plane. Rotation in counter-clockwise directions or translation of the toric IOL 200 may also be prevented by the geometry and/or orientation of the abutments 134.

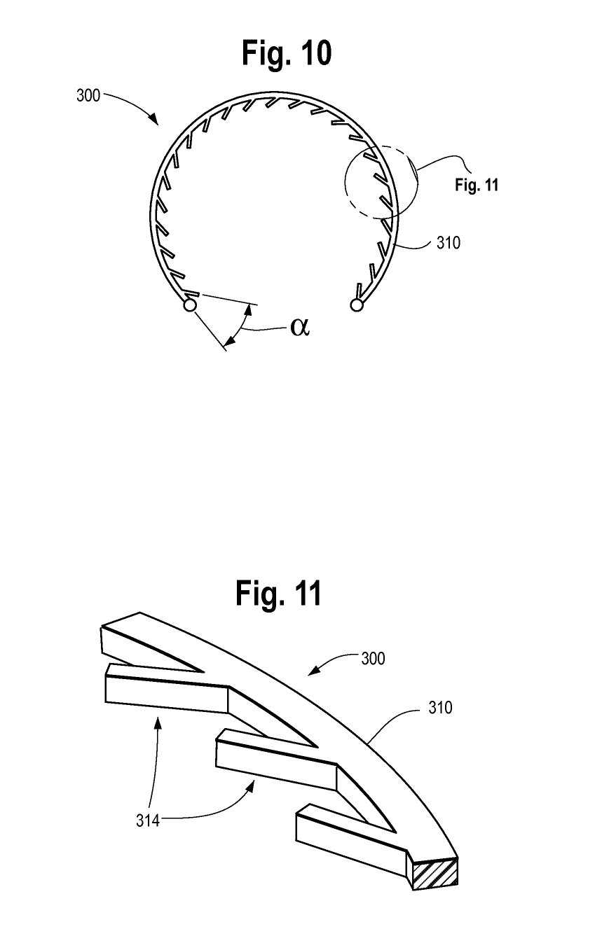

FIGS. 10-11 illustrate a second embodiment of a capsular tension apparatus or ring 300 according to the present invention. The second embodiment of the ring 300 functions in the same manner as the first embodiment of the ring 100 discussed above, except that the second embodiment of the ring 300 has a body 310 with abutments 314 in the form of angled cantilevers that are formed at an angle alpha with respect to the body 310. The angle alpha may be varied to suit a variety of mating anti-rotation features or haptics of a toricIOL.

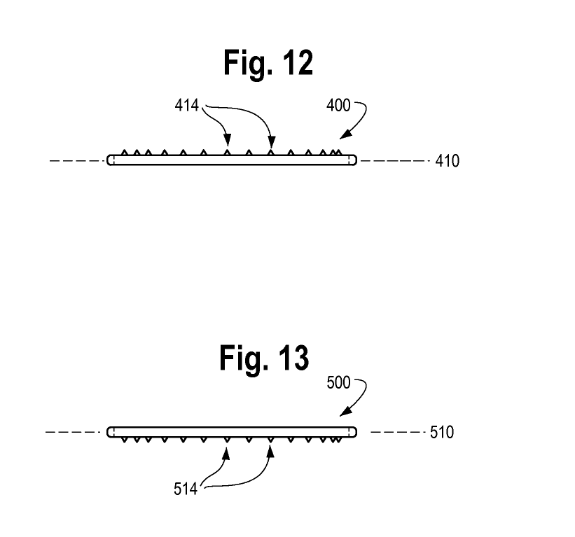

Another embodiment of a capsular tension apparatus or ring 400 according to the present invention is illustrated in FIG. 12. Ring 400 functions in the same manner as the first embodiment of the ring 100 discussed above, except that ring 400 has a plane 410 defined by the body of the ring 400 with abutments 414 extending upwardly away from the posterior chamber 46 of the eye 20 when the ring 400 is implanted into the eye 20. The angle of the abutments 414 with respect to the plane 410 may be varied to suit a variety of mating anti-rotation features or haptics of a toric IOL.

Another embodiment of a capsular tension apparatus or ring 500 according to the present invention is illustrated in FIG. 13. Ring 500 functions in the same manner as the first embodiment of the ring 100 discussed above, except that ring 500 has a plane 510 defined by the body of the ring 500 with abutments 514 extending downwardly toward the posterior chamber 46 of the eye 20 when the ring 500 is implanted into the eye 20. The angle of the abutments 514 with respect to the plane 510 may be varied to suit a variety of mating anti-rotation features or haptics of a toric IOL.

The method of implantation and operation of the inventive ring 100 will now be discussed. Rings 300,400, 500 function and would be implanted in the same manner as described hereinafter with respect to ring 100. After the capsular bag 42 has been incised and nucleus or lens 36 of the eye has been removed, the ring 100 may be implanted. The physician performing the implantation will provide the ring 100, which has a ring-like body 110 for stabilizing the periphery of the capsular bag 42 and further has an outer surface 130 with one or more abutments 134 extending therefrom. The physician performing the implantation will further provide the toric intraocular lens 70, 200, which has haptics 78, 208 that may have one or more anti-rotation projections 214 extending therefrom. The physician will inject OVD into the capsular bag 42 for improved manipulation during surgery. The physician then implants the ring 100 into the capsular bag 42 through the incision in the anterior chamber 40 of the eye 20. The physician then implants and aligns the toric intraocular lens 200 in the capsular bag 42. The OVD is then removed from the capsular bag 42 such that movement of the toric intraocular lens 70, 200 in the eye is inhibited by engagement of the abutments 134 with haptics 78 and/or the anti-rotation projections 214.

The illustrated preferred embodiments are included herein for descriptive purposes only and are not to be interpreted as limiting in any way of the broadest concepts of the present invention. It will be appreciated, however, that that novel surgical apparatuses and methods for implanting the same are not limited to the field of ophthalmology and may be used in surgical, or microsurgical, operations on other areas of the human or non-human body.

1. A surgical apparatus for implantation into organic tissue and for use with an implant, said implant having at least one stabilizing haptic, said surgical apparatus comprising:
a ring-like body for stabilizing organic tissue, said body having an outer surface and at least one abutment extending from said outer surface, said at least one abutment configured to engage the at least one stabilizing haptic of the implant to inhibit movement of the implant within the organic tissue.

2. The surgical apparatus of claim 1 wherein said surgical apparatus has the form of a capsular tension ring for stabilizing the capsule of the human eye, and the implant is an intraocular lens.

3. The surgical apparatus of claim 1 wherein said at least one abutment extends radially inward from said body outer surface.

4. The surgical apparatus of claim 1 wherein said body has a plurality of abutments extending from said body outer surface.

5. The surgical apparatus of claim 4 wherein said body has between about 18 and 36 abutments extending from said body outer surface.

6. The surgical apparatus of claim 1 wherein said at least one abutment extends from said body outer surface substantially in a plane defined by said body.

7. The surgical apparatus of claim 1 wherein said at least one abutment extends from said body outer surface substantially out of a plane defined by said body.

8. The surgical apparatus of claim 1 wherein said body further has a first end and a second end defining an opening therebetween.

9. The surgical apparatus of claim 8 wherein at least one of said first and second ends has an eyelet for manipulating said body during implantation of said surgical apparatus.

10. The surgical apparatus of claim 1 wherein said at least one abutment is defined by two intersecting surfaces to form a barb.

11. The surgical apparatus of claim 10 wherein at least one of said intersecting surfaces is arcuate.

12. The surgical apparatus of claim 1 wherein said at least one abutment inhibits clockwise rotation of the implant within the organic tissue.

13. The surgical apparatus of claim 1 wherein said at least one abutment inhibits counter-clockwise rotation of the implant within the organic tissue.

14. The surgical apparatus of claim 1 wherein said body outer surface is generally rounded.

15. A method of implanting a surgical apparatus into organic tissue, said method comprising the steps of:
a. providing a surgical apparatus, said surgical apparatus having a ring-like body, said body having an outer surface and at least one abutment extending from said outer surface;
b. providing an implant, said implant having at least one stabilizing haptic extending therefrom;
c. injecting a viscoelastic device into the organic tissue;
d. implanting said surgical apparatus within the organic tissue;
e. implanting said implant within the organic tissue to engage with said surgical apparatus; and
f. removing said viscoelastic device from organic tissue such that movement of said implant within the organic tissue is inhibited by engagement of said at least one stabilizing haptic with said at least one abutment of said surgical apparatus.
